(19) United States
(12) Patent Application Publication
(10) Pub. No.: US 2012/0302902 A1
(43) Pub. Date: Nov. 29, 2012

(54) Apparatus And Method For Diagnosis Of Individual Characteristics Based On Oscillometric Arterial Blood Pressure Measurement

(75) Inventor: Young Suk Shin, Gwangju (KR)

(73) Assignee: Industry-Academic Cooperation Foundation, Chosun University, Gwangju (KR)

(21) Appl. No.: 13/576,791

(22) PCT Filed: Apr. 20, 2011
(86) PCT No.: PCT/KR2011/002821

§ 371 Date: Aug. 2, 2012

(30) Foreign Application Priority Data
Jul. 12, 2010 (KR) 10-2010-0066725
Apr. 15, 2011 (KR) 10-2011-0035405

(51) Int. Cl. A61B 5/025; A61B 5/022

(52) U.S. Cl. 600/494

(57) ABSTRACT

This invention relates to a personal feature diagnostic apparatus on the basis of an oscillometric arterial pressure measurement comprising a pressure detection sensor unit for detecting a cuff pressure including an effect of pulse wave; a pulse wave extraction unit for extracting pulse wave signals of the artery from the cuff pressure detected in the pressure detection sensor unit; a pulse wave amplitude detection unit for dividing pulse wave signals extracted from the pulse wave extraction unit into a plurality of window sections and detecting a minimum amplitude pulse in each window section; and a personal feature diagnostic unit for diagnosing personal feature by using the minimum amplitude pulse in each window section detected by the pulse wave amplitude detection unit.

### FIELD OF THE INVENTION

The invention relates to a apparatus and method for diagnosing personal features based on oscillometric arterial pressure measurement, and specifically to an apparatus and method for diagnosing personal features, using minimum amplitude values respectively detected from pulse wave signals corresponding to a plurality of window sections.

### BACKGROUND OF THE INVENTION

Various researches for providing medical services tailored to personal features have been actively made. If personal features are diagnosed using a blood pressure, it would be useful in providing treatment tailored to subjects having a blood pressure, a kidney disease, or diabetes.

Generally, a method for measuring a blood pressure is classified into an invasive method and non-invasive method. The invasive method is a direct measurement method which inserts a cannular needle into the interior of the artery, so that the subject is reluctant or is susceptible to infection. Meanwhile, the non-invasive method is classified into an auscultation method and an oscillometric method. The oscillometric method was developed because the auscultation method has a problem that the auscultation method is not suitable for long running measurement and can be performed by the expert only. The oscillometric method is similar to the auscultation method, but it uses a pressure sensor instead of a stethoscope in order to register pressure oscillation in the inside of a cuff.

However, an oscillometric arterial pressure measurement up to now have not considered personal features and have focused on exactly measuring a high blood and a low blood pressure of the subject. Therefore, the blood pressure measuring method in the prior art which did not consider personal features was not able to detect an abnormal pattern of a blood pressure originated from diseases, etc.

### SUMMARY OF THE INVENTION

To solve the above problems, the object of this invention is to provide a personal feature diagnostic apparatus and method based on an oscillometric arterial pressure measurement for diagnosing personal features by using minimum amplitude values each detected from pulse wave signals corresponding to a plurality of window sections

In addition, another personal feature diagnostic apparatus and method based on an oscillometric arterial pressure measurement for diagnosing personal features by means of averaging out a plurality of normalized pattern signals of the subject obtained by using a plurality of pulse wave signals obtained from the same subject and normalizing the pulse wave signals.

Further, yet another object of this invention is to provide an apparatus and method for diagnosing personal features based on an oscillometric arterial pressure measurement by averaging out a plurality of normalized pattern signals of the subject.

To solve the above problems, personal feature diagnostic apparatus on the basis of an oscillometric arterial pressure according to this invention comprises: a pressure detection sensor unit for detecting a cuff pressure including an effect of pulse wave; a pulse wave extraction unit for extracting pulse wave signals of the artery from the cuff pressure detected in the pressure detection sensor unit; a pulse wave amplitude detection unit for dividing pulse wave signals extracted from the pulse wave extraction unit into a plurality of window sections and detecting a minimum amplitude pulse in each window section; and a personal feature diagnostic unit for diagnosing personal feature by using the minimum amplitude pulse in each window section detected by the pulse wave amplitude detection unit.

Preferably, the apparatus further comprises a pulse wave signal normalization unit for generating normalized pulse wave signals by using a plurality of pulse wave signals extracted from the pulse wave extraction unit obtained from the same subject to output the normalized pulse wave signals into the pulse wave amplitude detection unit wherein the pulse wave amplitude detection unit divides the normalized pulse wave signals outputted from the pulse wave signal normalization unit into a plurality of window sections and detects a minimum amplitude pulse in each window section.

It is preferable that the personal feature diagnostic unit diagnoses personal features by averaging out a minimum amplitude pulse in each window section detected from the pulse wave amplitude detection unit per the corresponding window section per each subject.

It is preferable that the pulse wave signal normalization unit comprises normalizing by using root value of sum of square of each of a plurality of pulse wave signals extracted from the pulse wave extracting unit.

It is preferable that the number of window at the time of dividing pulse wave signals extracted from the pulse wave extraction unit includes a minimum oscillometric waveform number and a pulse number of one turn in a maximum period.

It is preferable that the personal feature diagnostic unit comprises diagnosing the subject as having a high blood pressure if the largest value of a minimum amplitude pulse or an average minimum amplitude pulse in each window section extracted by the pulse wave amplitude detection unit is within ⅓ of a measurement period.

It is preferable that the personal feature diagnostic unit comprises diagnosing personal features using a minimum amplitude pulse and a maximum amplitude pulse in each window section detected by the pulse wave amplitude detection unit.

To solve the above problems, a personal feature diagnosing method on the basis of an oscillometric arterial pressure measurement according to this invention comprises steps of: detecting a cuff pressure including an effect of pulse wave from a cuff wound on the arm or the wrist of the subject; extracting pulse wave signals of the artery from the extracted cuff pressure; dividing the extracted pulse wave signals into a plurality of window sections and detecting a minimum amplitude pulse in each window section; and diagnosing personal features using a minimum amplitude pulse in the each window section.

To solve the above problems, personal feature diagnostic apparatus on the basis of an oscillometric arterial pressure according to this invention comprises: a pressure detection sensor unit for detecting a cuff pressure including an effect of pulse wave; a pulse wave extraction unit for extracting pulse wave signals of the artery from the cuff pressure detected in the pressure detection sensor unit; a pulse wave amplitude detection unit for dividing pulse wave signals extracted from the pulse wave extraction unit into a plurality of window sections and detecting a minimum amplitude pulse in each window section; a personal pattern generating unit for generating personal pattern by using the minimum amplitude pulse in each window section detected by the pulse wave amplitude detection unit; a storing unit for storing personal feature pattern generated by the personal pattern generating unit; and a personal feature variation diagnostic unit for diagnosing personal feature variation by comparing personal feature pattern stored in the storing unit with feature pattern measured using cuff pressure of cuff wound on the arm or wrist of the subject.

To solve the problems, a personal feature diagnosing method on the basis of an oscillometric arterial pressure measurement according to this invention comprises steps of: detecting a cuff pressure including an effect of pulse wave from a cuff wound on the arm or the wrist of the subject; extracting pulse wave signals of the artery from the extracted cuff pressure; dividing the extracted pulse wave signals into a plurality of window sections and detecting a minimum amplitude pulse in each window section; generating personal feature pattern using a minimum amplitude pulse in the each window section; storing the generated personal feature pattern; and diagnosing personal feature variation by comparing the stored personal feature pattern with feature pattern measured using cuff pressure of cuff wound on the arm or wrist of the subject.

According to the above constructions and their features of this invention, this invention can diagnose personal features using pulse wave information at the time of blood pressure measurement.

In addition, according to this invention it is possible to exactly diagnose personal features by using and normalizing a plurality of pulse wave signals obtained from the same subject to reduce the variation of pulse wave signals which occurs each time the same subject is measured.

Further, according to this invention it is possible to reduce a measurement error by averaging out minimum amplitude values and/or maximum amplitude values to diagnose personal features.

### BRIEF DESCRIPTION OF THE DRAWINGS

FIG. 1 shows an apparatus for diagnosing a personal feature on the base of oscillometric arterial pressure measurement according to one embodiment of the invention.

FIG. 2 is a flowchart showing personal feature diagnosing method on the basis of an oscillometric arterial pressure measurement shown in FIG. 1.

FIG. 3 shows data measured over 5 turns each for a plurality of subjects.

FIG. 4 shows a pressure signal of the cuff shown in FIG. 1.

FIG. 5 shows pulse wave signals extracted from pulse wave extraction unit shown in FIG. 1.

FIG. 6 shows a minimum amplitude value and a maximum amplitude value in each window section.

FIG. 7 shows a minimum amplitude value detected in each window using a minimum amplitude value and a normalized pulse wave signal in each window of the same subject.

FIG. 8 shows personal feature pattern belonging in a high blood pressure class at systolic blood pressure among the subjects in FIG. 3.

FIG. 9 shows personal feature pattern belonging in a normal blood pressure class at systolic blood pressure among the subjects in FIG. 3.

FIG. 10 shows a method for diagnosing personal features on the base of oscillometric arterial pressure measurement according to another embodiment of the invention shown in FIG. 2.

FIG. 11 shows a minimum amplitude value and a maximum amplitude value detected in each window of the same subject.

FIG. 12 shows personal feature pattern belonging in a high blood pressure class at diastolic blood pressure among the subjects in FIG. 3.

FIG. 13 shows personal feature pattern belonging in a normal blood pressure class at diastolic blood pressure among the subjects in FIG. 3.

FIG. 14 shows a personal feature diagnostic apparatus based on an oscillometric arterial pressure measurement according to another embodiment of this invention.

FIG. 15 is a flow chart of personal feature diagnosing method based on an osillometric arterial pressure measurement shown in FIG. 14.

### DETAILED DESCRIPTION OF THE PREFERRED EMBODIMENT

From now on, a apparatus an method for diagnosing an personal feature on the base of oscillometric arterial pressure measurement according to embodiments of the invention will be explained in detail, referring to the accompanying drawings.

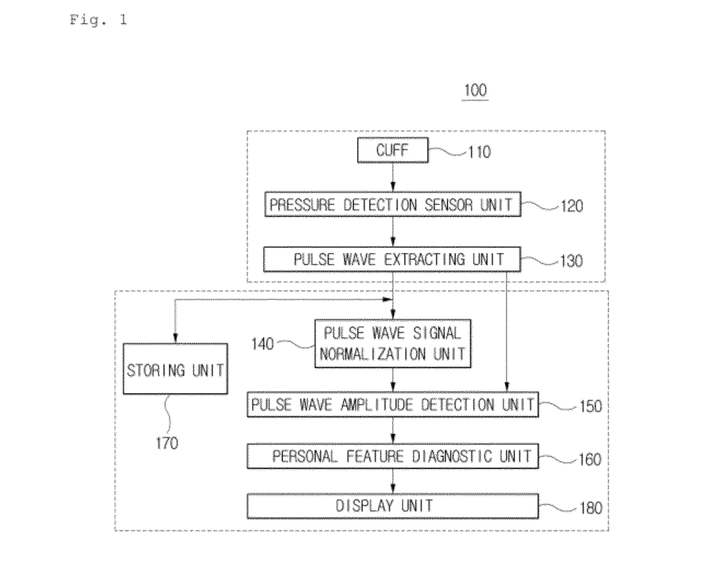

FIG. 1 shows a apparatus for diagnosing an personal feature on the base of oscillometric arterial pressure measurement according to one embodiment of the invention.

Whenever the heart beats, it pushes the blood through the artery. This flow of the blood makes the artery expand and contract repeatedly, which is known as a pulse(s). An oscillometric method, which is one of blood pressure measurement methods, generally comprises linearly or gradually measuring a pressure of the blood vessel of arms, etc., at contraction and expansion of the heart, extracting a pulse wave signal from a measured pressure and obtaining a maximum blood pressure (systolic blood pressure) and a minimum blood pressure (diastolic blood pressure).

As shown in FIG. 1, one embodiment of a apparatus for diagnosing an personal feature based on oscillometric arterial pressure measurement includes a cuff 100, a pressure detection sensor unit 120, a pulse wave extraction unit 130, a pulse wave signal normalization unit 140, a pulse wave amplitude detection unit 150, an personal feature diagnostic unit 160, a storage unit 170, and a display unit 180.

The cuff 110, which is wound at a measurement portion such as arms or forearms at the time of measuring the blood pressure, is connected with a pressurizing pump (not shown) and a discharge valve (not shown) through an inner conduit (not shown).

A pressure detection unit 120 is connected with the cuff 110 and thus detects a cuff pressure including the effect of pulse wave, i.e., a pressure of cuff 110.

A pulse wave extraction unit 130 extracts a pulse wave signal of the artery of all sections from cuff pressure detected at the pressure detection unit 120. In the meantime, when a plurality of pulse wave signals are intended to be obtained from the same subject to normalize a pulse wave signal, the pulse wave signal extracted from pulse wave extraction unit 130 is stored into a pulse wave signal storing area of a storing unit 170.

Pulse wave signal normalization unit 140 normalizes a plurality of pulse wave signals obtained from the same subject extracted from the pulse wave extraction unit 130, to reduce a variation in wave pulse signals wherein the variation is shown per each number of the measurement of the same subject.

Pulse wave amplitude detection unit 150 divides pulse wave signal inputted to pulse wave extraction unit 130 or normalized pulse wave signal of the subject into a plurality of window sections and detects a minimum amplitude pulse (MIAP) and a maximum amplitude pulse (MXAP) respectively per each window section.

Personal feature diagnostic unit 160 diagnoses personal feature, using a minimum amplitude pulse and a maximum amplitude pulse in each window section detected by the pulse wave amplitude detection unit 150.

Storing unit 170 can be used when a plurality of pulse wave signals are intended to be obtained from the same subject to normalize pulse wave signal. In this case pulse wave signals extracted from pulse wave extraction unit 130 are stored in a pulse wave signal storing section of the storing unit 170.

Display unit 180 can indicate personal feature information such as high blood pressure data, kidney diseases and diabetes of the subject.

Meanwhile, although personal feature diagnostic unit 100 shown in FIG. 1 may be implemented as one apparatus such as a blood pressure gauge, the personal feature diagnostic unit 100 may also be implemented as being divided into a blood pressure gauge including a cuff 100, a pressure detection sensor unit 120 and pulse wave extraction unit 130 and a computer/monitor including a pulse wave signal normalization unit 140, a pulse wave amplitude detection unit 150, a personal feature diagnostic unit 160, a storing unit 170 and a display unit 180.

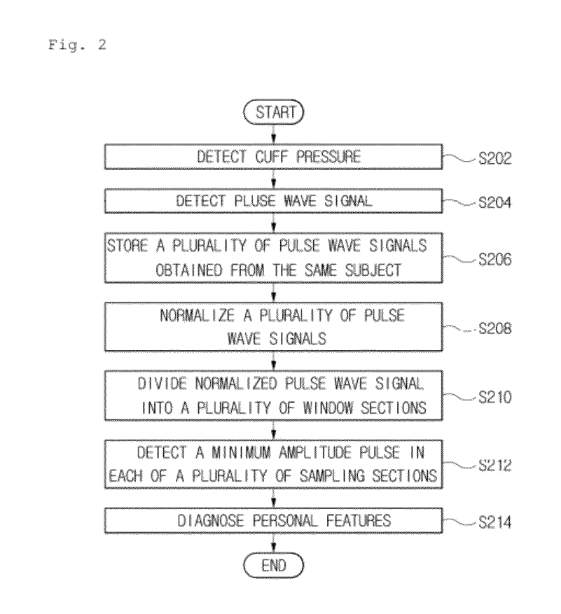

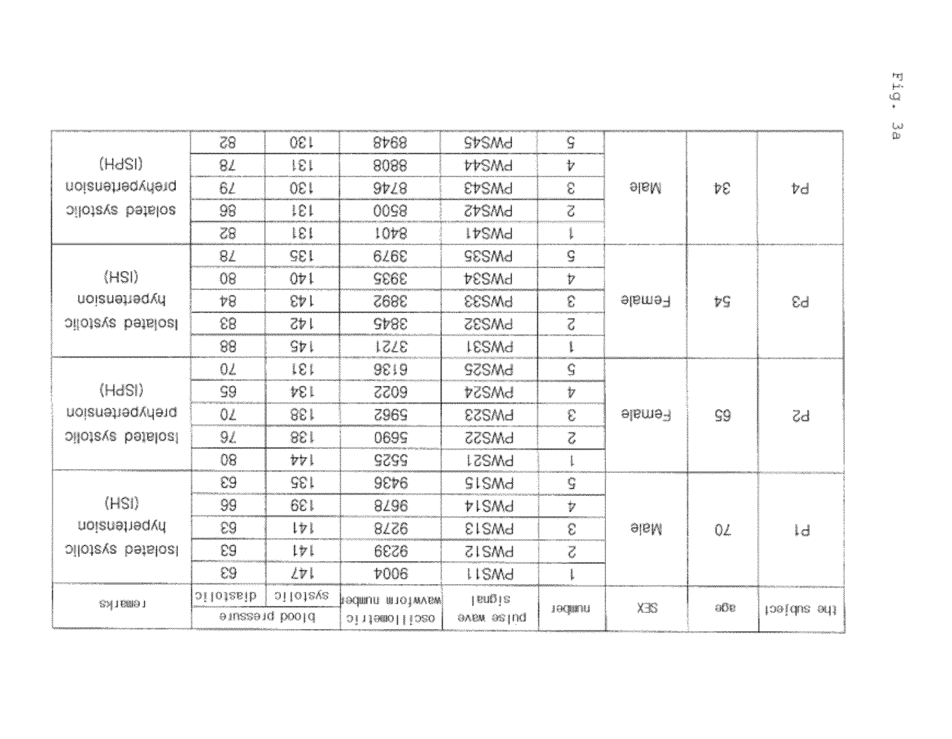

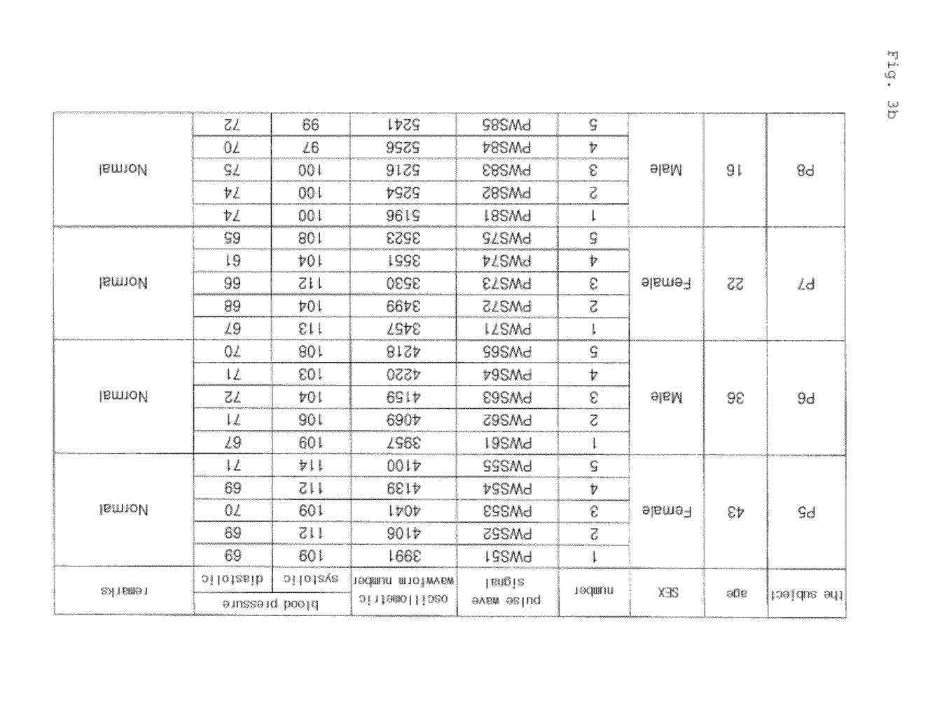

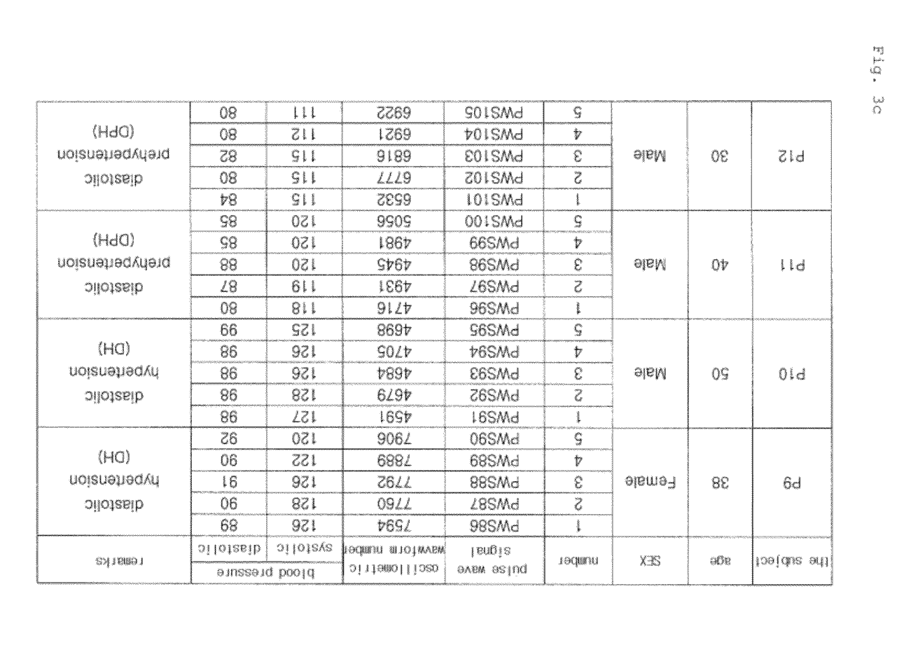

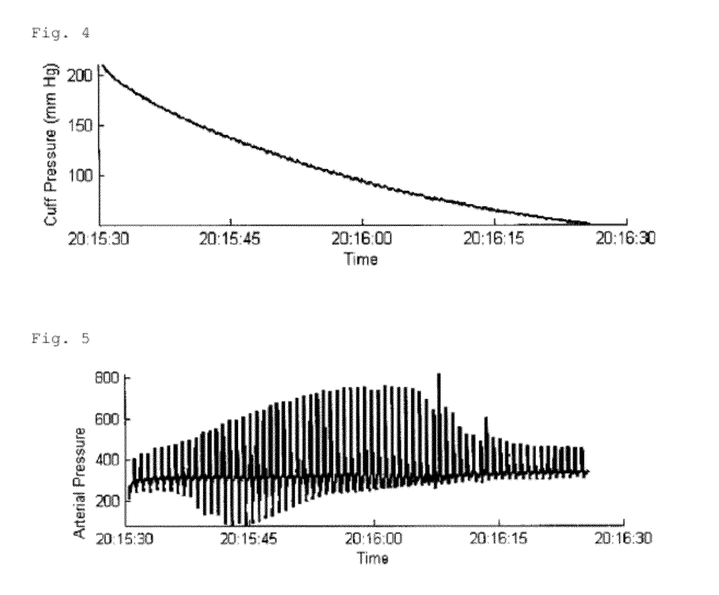

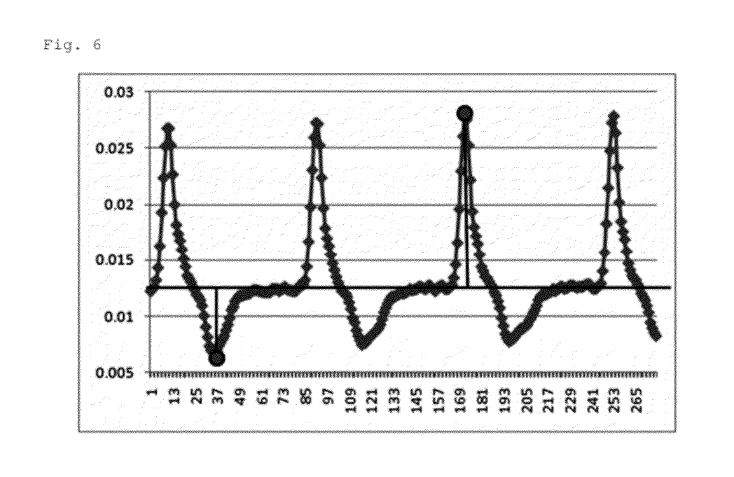

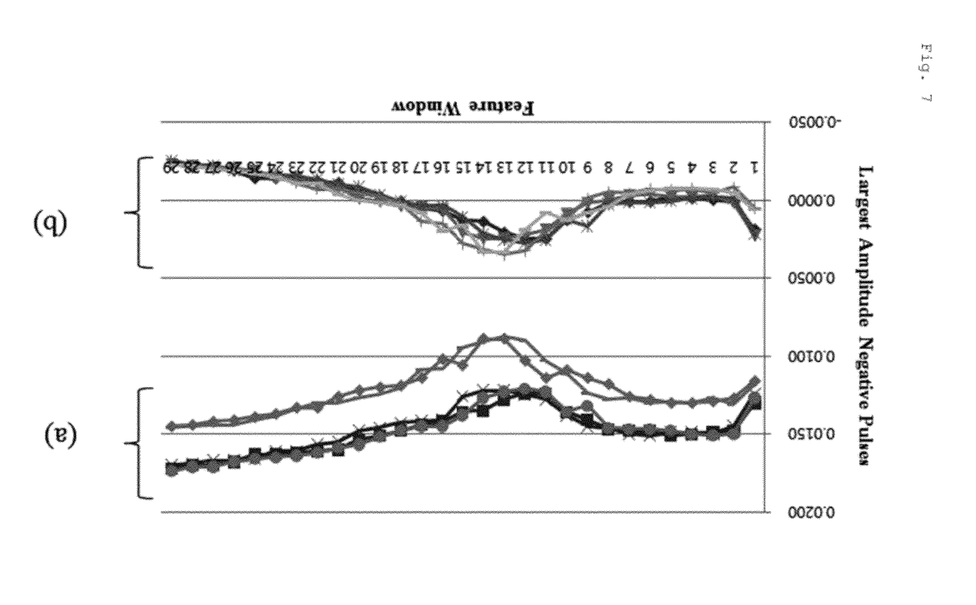

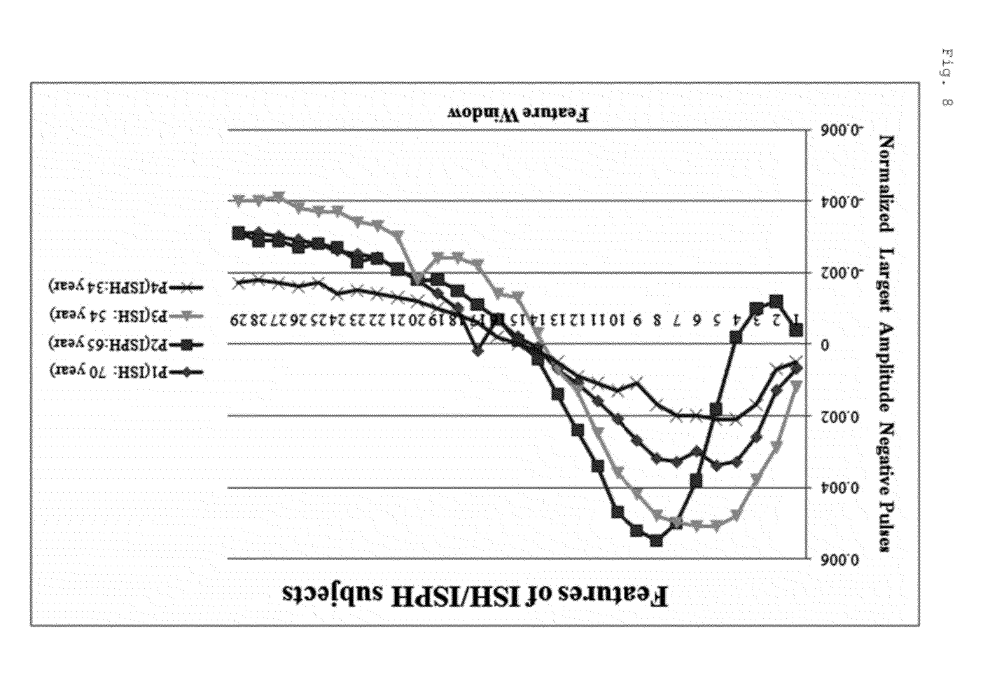

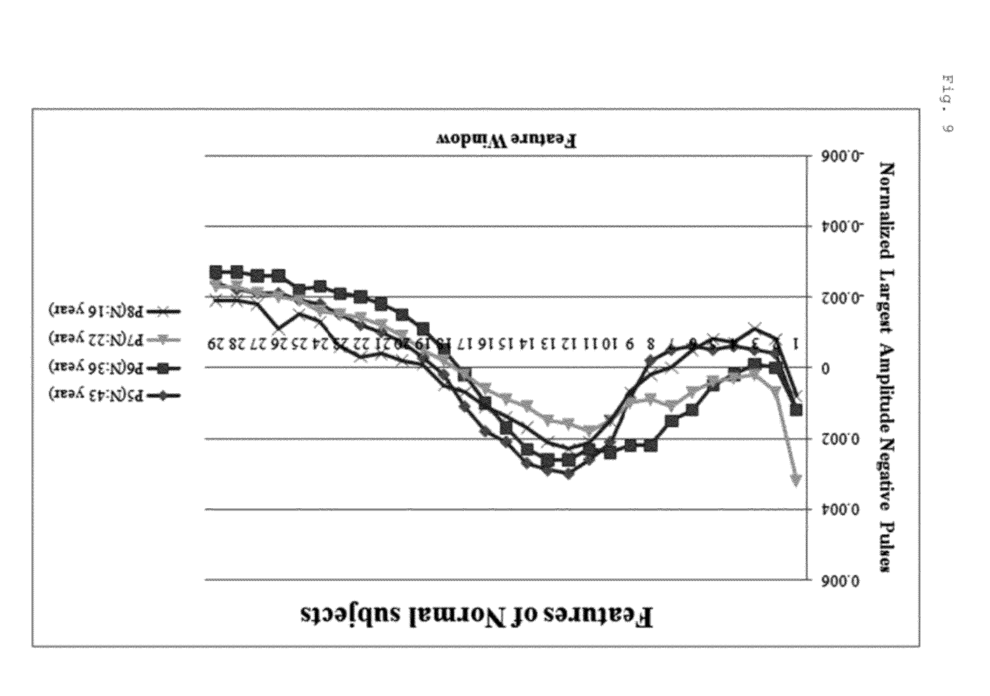

FIG. 2 is a flow chart showing a personal feature diagnosing method based on an oscillometric arterial pressure measurement of the apparatus shown in FIG. 1. FIGS. 3a to 3c are data sheets in which data measured over five numbers for a plurality of subjects respectively are written. FIG. 4 shows a cuff pressure of the cuff shown in FIG. 1. FIG. 5 is a drawing showing pulse wave signals extracted from the pulse wave extraction unit of FIG. 1. FIG. 6 shows a minimum amplitude value and a maximum amplitude value in one window section. FIG. 7 is a drawing showing a minimum amplitude pulse detected in each window of the same measuring person and a minimum amplitude pulse detected in each window using a normalized pulse wave signal. FIG. 8 is a drawing showing personal feature patterns belonging in a high blood pressure group in systolic blood pressure among the subjects shown in FIG. 3. FIG. 9 is a drawing showing personal feature patterns belonging in a normal blood pressure group in systolic blood pressure among the subjects shown in FIG. 3.

With reference to FIG. 2, a flow chart and steps for diagnosing personal features on the basis of an oscillometric arterial pressure measurement will be explained below.

Cuff 110 is wound on arms or wrists and if a personal feature diagnostic unit 100 operates to measure a blood pressure a cuff pressure is detected via a pressure detection sensor unit 120 (S202). A cuff pressure detected in the pressure detection sensor unit 120 is shown in FIG. 4.

Pulse wave signals of the artery in all sections are extracted at pulse wave extraction unit 130 from a cuff pressure detected from the pressure detection sensor unit 120 (S204). Pulse wave signal extracted in pulse wave extraction unit 130 is shown in FIG. 5. This pulse wave signal can be extracted by using differential information of a cuff pressure being sequentially inputted from the pressure detection sensor unit 120 and removing the effect of a mean depressurization amount of a cuff pressure included in differential information.

Pulse wave signals extracted from pulse wave extraction unit 120 can be stored in a storing unit 170 until a plurality of pulse wave signals are obtained from the same subject (S206). For each subject data measured over five numbers are shown in FIG. 3.

That is to say, pulse wave signal extracted from a cuff pressure and the number of oscillometric waveform of pulse wave signal are stored in a storing unit 170, together with age, sex and a maximum blood pressure (systolic blood pressure) and a minimum blood pressure (diastolic blood pressure) detected via a stethoscope by a nurse. In addition, each data from each the same subjects, in other words over many times the pulse wave signal extracted from pulse wave extraction unit 130, pulse wave signal extracted from a cuff pressure, and a maximum blood pressure and a minimum blood pressure detected via a stethoscope are stored in a storing unit 170.

To obtain satisfactory personal feature information in the invention, a measuring room is preferably constructed such that an exact blood pressure can be measured and the subjects also preferably take a sufficient rest in a waiting room, etc. Data of table shown in FIG. 3 are ones repeatedly obtained with a period of one minute measurement and one minute rest after taking a rest in a waiting room but data can be obtained randomly after taking a rest.

It is understood from FIG. 3 that the number of oscillometric waveform measured over five numbers is similar for the same subjects. This means that the same subject exhibits pulse wave signals of a similar size.

If the measurement number for the same subject reaches N number, a pulse wave signal normalization unit 140 performs normalization in order to reduce a variation in pulse wave signals showing a variation per each measurement number of the same subject (S208). That is to say, in order to obtain data showing that personal features can be more in detailed diagnosed on the basis of an oscillometric arterial pressure measurement, a plurality of pulse wave signals for the same subject stored in a storing unit 170 are normalized.

Pulse wave signal corresponding to the subject can be normalized through normalization in the pulse wave signal normalization unit 140 on the basis of the following formula 1:

First, the number of total data (=60 in FIG. 3) obtained is expressed as W and the number of the subject (in FIG. 3 the number of the subject is 12 persons) is expressed as N. Therefore, the following can be obtained.

W={Wᵢ}ᵢ₌₁^{N}

In addition, pulse wave signals obtained from the same subjects can expressed as Wᵢ={Wᵢⱼ}ⱼ₌₁^{N}^{ᵢ}
Pulse wave signal (Wᵢⱼ) means the jth pulse wave signal of the ith subject.

$$φ = sqrt ( {\sum }_{j = 1}^{{N}_{i}} {( {w}_{ij} )}^{2} ) \\ {W}_{i}^{*} = {W}_{i} / φ \qquad [ FORMULA 1 ]$$

where pulse wave size φ of the same subject is a root of sum of squares of pulse wave signals of the same subject, normalized pulse wave signal Wi* is normalized pulse wave signals obtained by dividing pulse wave signals of the subject by pulse wave size φ.

Pulse wave amplitude detection unit 150 divides all sections of normalized pulse wave signal obtained from pulse wave signal normalization unit 140 into a plurality of window sections (S210). In this case, the number of feature window is defined on the basis of a minimum oscillometric waveform number of normalized pulse wave signal of each subject (in the case of the subject S1 in FIG. 3, the minimum oscillometric waveform number is 9004) and oscillometric waveform number generated from one turn of pulsation. In addition, to compare features between mapping windows of the subject to be diagnosed, all normalized pulse wave signals are preferable to be divided by th same window number.

The number of feature window (FW) can be determined by two parameters. If a minimum oscillometric waveform number is expressed as α, oscillometric waveform number of pulse including one turn of pulse during a maximum period is expressed as β, the number of feature window can be calculated by the following formula 2:

α=min{Wᵢⱼ}^{N}^{ᵢ} [FORMULA 2]

FW=α/β

In the invention, using formula 2, all sections of normalized pulse wave pattern obtained in pulse wave signal normalization unit 140 are divided into 29 window sections.

Pulse wave amplitude detection unit 150 detects minimum amplitude values respectively in a plurality of window sections. Respective minimum amplitude values in one window section are shown in FIG. 6. In pulse wave signal shown in FIG. 6 an upper end region indicates a maximum amplitude value and a lower end region indicates a minimum amplitude value. Therefore, at least one number of pulse wave signals should be included in one window section and when a plural number of pulse wave signals are in one window section the smallest value thereof becomes a minimum amplitude value. Although a minimum amplitude value is here described to is detected, a maximum amplitude value or a mean amplitude value (MAP: Mean Amplitude Pulse) obtained by equating amplitude values in one window section according to personal features to be diagnosed, as well as a minimum amplitude value only, can be included to understand personal features.

In the invention, a minimum amplitude value in each window is detected by dividing all sections of normalized pulse wave pattern obtained in the pulse wave signal normalization unit 140 into 29 window sections. Minimum amplitude values detected in each window of the same subject are shown in FIG. 7(a) and minimum amplitude values which use normalized pulse wave signal in the pulse wave signal normalization unit 140 are shown in FIG. 7(b).

Personal feature diagnostic unit 160 diagnoses personal features using a minimum amplitude value in each window section detected by pulse wave amplitude detection unit 150 (S214). Preferably, for each of the subjects, personal feature diagnostic unit 160 diagnoses personal features by averaging out minimum amplitude values in each window section detected by pulse wave amplitude detection unit 150 per the corresponding window section. This average minimum amplitude values are indicated in personal feature patterns in FIGS. 8 and 9. FIG. 8 shows personal feature patterns of the subjects (P1 to P4) diagnosed as a high blood pressure at systolic blood pressure and FIG. 9 shows personal feature patterns of the subjects (P5 to P8) diagnosed as a normal blood pressure at systolic blood pressure.

As known in FIGS. 8 and 9, in the case of the subjects diagnosed as having a high blood pressure, the largest value among the average minimum amplitude values in each window section detected by pulse wave amplitude detection unit 150 is within ⅓ of a measurement period.

It will be understood that a apparatus and method for diagnosing personal features on the basis of oscillometric arterial pressure measurement mentioned above can be used effectively through the table 1.

TABLE 1

| | Systolic blood | Systolic blood |
|---|---|---|
| The | pressure (mmHg) | pressure (mmHg) converted |
| subject | shown in FIG. 3 | using a neural network |
| P1 | 139 | 133 |
| | 135 | 145 |
| P2 | 134 | 133 |
| | 131 | 127 |
| P3 | 140 | 134 |
| | 135 | 121 |
| P4 | 131 | 124 |
| | 130 | 128 |
| P5 | 112 | 113 |
| | 114 | 109 |
| P6 | 103 | 107 |
| | 108 | 107 |
| P7 | 104 | 105 |
| | 108 | 98 |
| P8 | 97 | 92 |
| | 99 | 94 |

The first row of table 1 indicates the subjects, the second row thereof indicates systolic blood pressure (mmHg) in FIG. 3 measured by a nurse and the third row thereof indicates systolic blood pressure when the largest value of minimum amplitude values in each window section detected by the pulse wave amplitude detection unit 150 is inversely converted using a neural network.

As known in table 1, there is a little error between systolic blood pressure in FIG. 3 measured by a nurse and systolic blood pressure converted a neural network, but as a general measuring method of oscillometric arterial pressure by a blood pressure gauge also calculates systolic blood pressure and diastolic blood pressure from a median, an error range is relatively large. Therefore, the invention can be applied and such error can be reduced by a manner in which measurement data with a large deviation are removed from a number of measurement data.

Therefore, it is most useful to diagnose personal features using a minimum amplitude value and a mean minimum amplitude value in each window section detected by pulse wave amplitude detection unit according to the invention.

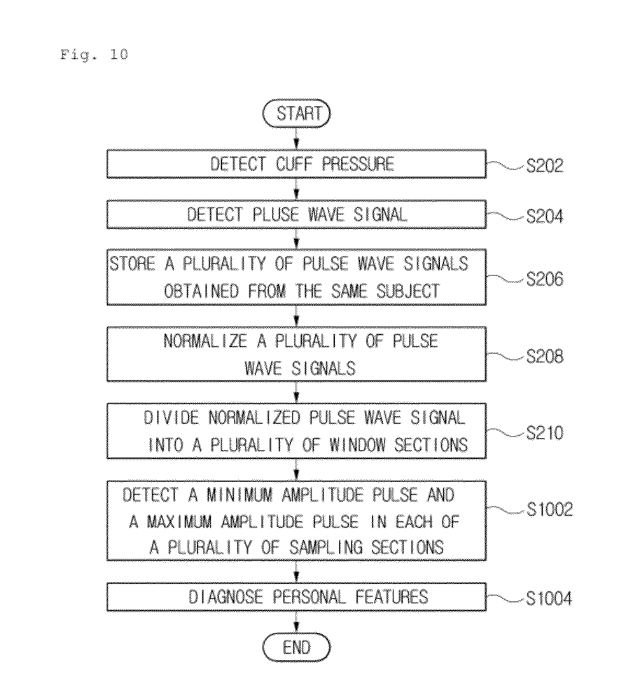

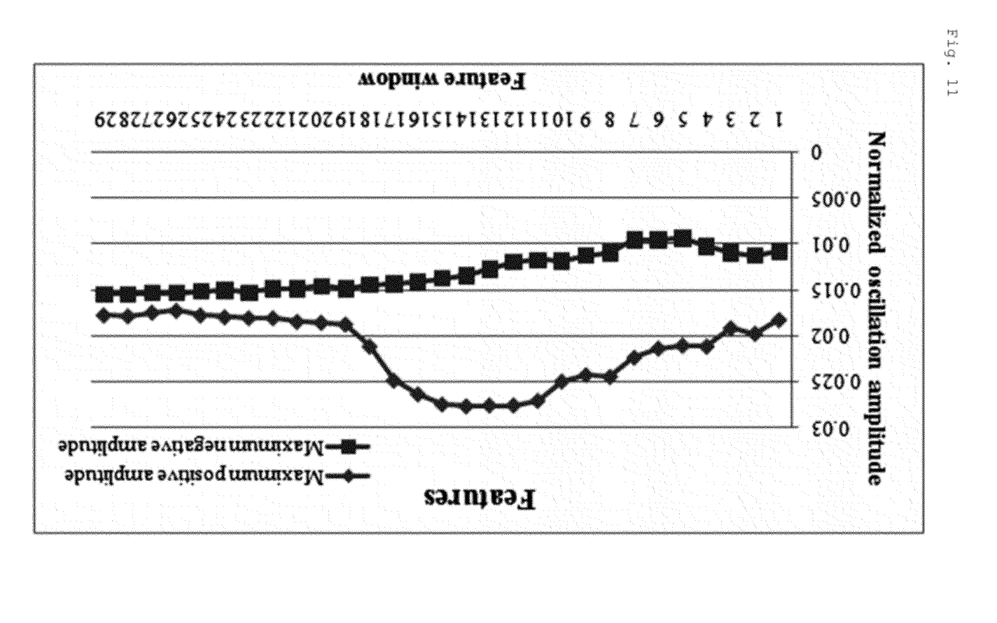

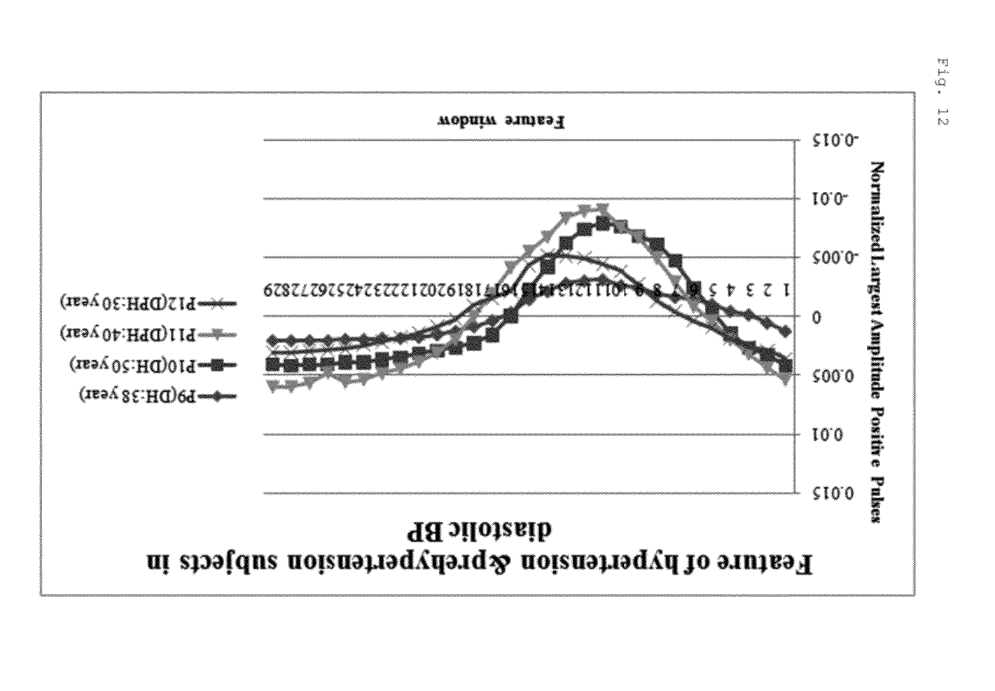

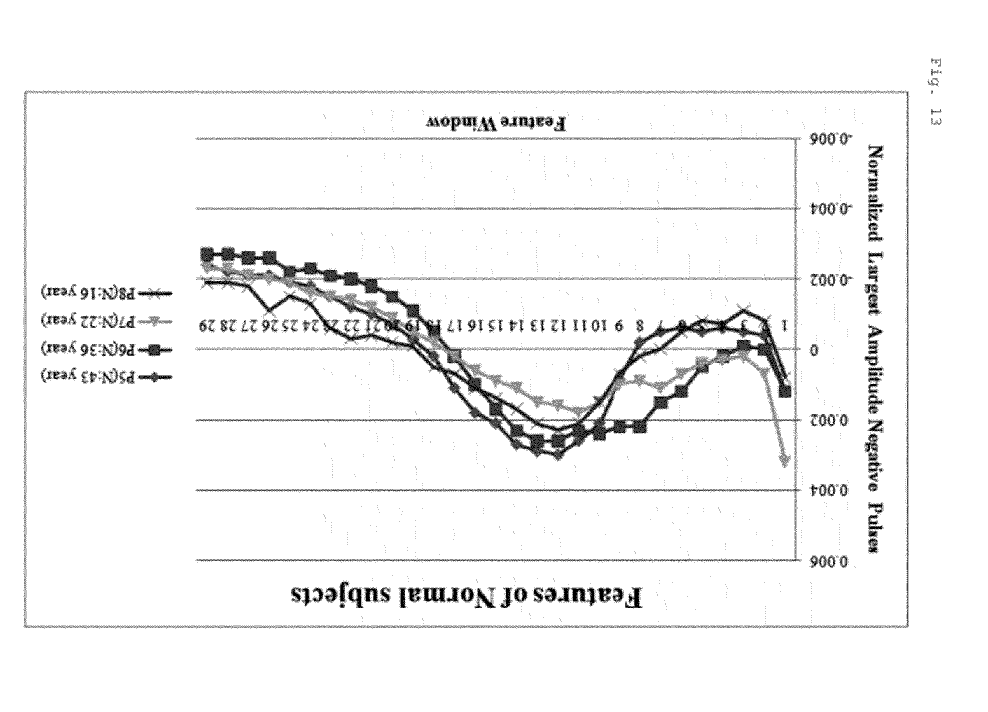

FIG. 10 is a flow chart according to embodiment 2 of a method of diagnosing personal features on the basis of oscillometric arterial pressure measurement shown in FIG. 1. FIG. 11 shows a minimum amplitude value and a maximum amplitude value in each window of the same measuring person. FIG. 12 shows personal feature patterns of persons belonging in high blood pressure group at diastolic blood pressure among the subjects shown in FIG. 3. FIG. 13 shows personal feature patterns of persons belonging in normal blood pressure group at diastolic blood pressure among the subjects shown in FIG. 3.

In the flow chart of FIG. 10 from step S202 of detecting cuff pressure to step S210 of dividing a normalized pulse wave signal into a plurality of window sections are the same as those of the flow chart shown in FIG. 2.

And then, pulse wave amplitude detection unit 150 detects a minimum amplitude value and a maximum amplitude value respectively in a plurality of window sections (S1002). As already explained above, FIG. 6 shows a minimum amplitude value and a maximum amplitude value in one window section. In pulse wave signal shown in FIG. 6 an upper end region indicates a maximum amplitude value and a lower end region indicates a minimum amplitude value. Therefore, at least one number of pulse wave signals should be included in one window section and when a plural number of pulse wave signals are in one window section the smallest value thereof becomes a minimum amplitude value. Meanwhile, pulse wave amplitude detection unit 150 can further obtain a mean amplitude value by averaging out amplitude values in one window section according to personal features to be diagnosed.

In the invention all sections of normalized pulse wave patterns obtained in pulse wave signal normalization unit 140 are divided into 29 sections to detect a minimum amplitude value and a maximum amplitude value in each window. A minimum amplitude value and a maximum amplitude value in each window of the same subject are indicated in FIG. 13.

Personal features are diagnosed using a minimum amplitude value and a maximum amplitude value in each window section detected by pulse wave amplitude detection unit 150. Preferably, personal feature diagnostic unit 160 diagnoses personal features by averaging out minimum amplitude values and maximum amplitude values in each window section per corresponding window section detected by pulse wave amplitude detection unit 150 per the subject. As already explained, these averaged minimum amplitudes are indicated in FIGS. 8 and 9 in the personal feature pattern. In FIG. 8 personal feature patterns of subjects (P1 to P4) diagnosed systolic blood pressure as a high one are indicated and in FIG. 9 personal feature patterns of subjects (P5 to P8) diagnosed as normal are indicated. In addition, these averaged maximum amplitude values are indicated in the personal feature pattern in FIGS. 12 and 13. In FIG. 12 personal feature patterns of subjects (P9 to P12) diagnosed a diastolic blood pressure as high blood pressure (90 to 99 mmHg) and suspected blood pressure (80 to 89 mmHg) are indicated and in FIG. 13 personal feature patterns of subjects (P5 to P8) diagnosed as normal blood pressure are indicated.

As already explained, in the case of the subjects diagnosed as having a systolic high blood pressure in FIG. 8 differently from the ones diagnosed as normal in FIG. 9, the largest value among the average minimum amplitude values in each window section detected by pulse wave amplitude detection unit 150 is within ⅓ of a measurement period. In addition, as known in FIGS. 12 and 13 in the case of in the case of the subjects diagnosed as having a diastolic high blood pressure in FIG. 12 differently from the ones diagnosed as normal in FIG. 13, the smallest value among the average maximum amplitude values in each window section detected by pulse wave amplitude detection unit 150 is focused between window 10 and 14 of a measurement period.

Accordingly, personal features can be diagnosed more in detailed if a maximum amplitude value or an average maximum amplitude value in each window section as well as a minimum amplitude value or an average minimum amplitude value in each window section detected by pulse wave amplitude detection unit according to the invention is used.

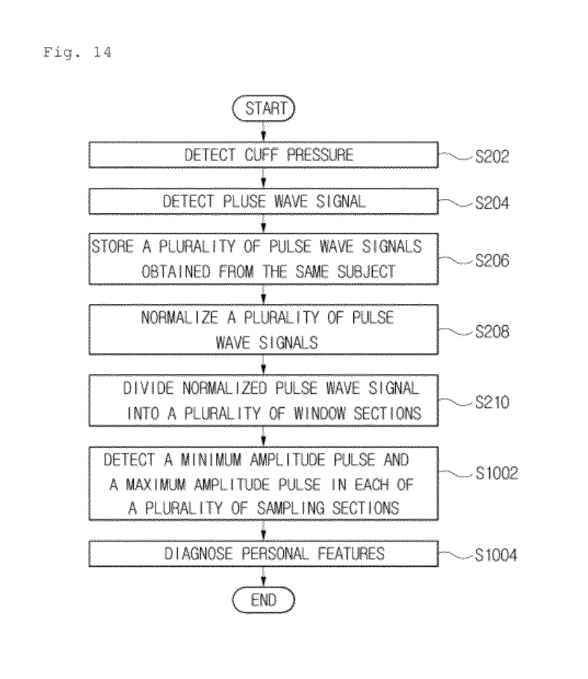

FIG. 14 shows a personal feature diagnostic apparatus based on an oscillometric arterial pressure measurement according to another embodiment of the invention.

As shown in FIG. 14, a personal feature diagnostic apparatus 100 comprises further a personal pattern generating unit 1410, a storing unit 1420 and a personal feature variation diagnostic unit 1430 in addition to a cuff 110, a pressure detection sensor unit 120, a pulse wave extraction unit 130 and a pulse wave signal normalization unit 140, a pulse wave amplitude detection unit 150 and a display unit 180.

The cuff 110, which is wound at a measurement portion such as arms or forearms at the time of measuring the blood pressure, is connected with a pressurizing pump (not shown) and a discharge valve (not shown) through an inner conduit (not shown).

A pressure detection unit 120 is connected with the cuff 110 and thus detects a cuff pressure including the effect of pulse wave, i.e., a pressure of cuff 110.

A pulse wave extraction unit 130 extracts a pulse wave signal of the artery of all sections from cuff pressure detected at the pressure detection unit 120. In the meantime, when a plurality of pulse wave signals are intended to be obtained from the same subject to normalize a pulse wave signal, the pulse wave signal extracted from pulse wave extraction unit 130 is stored into a pulse wave signal storing area of a storing unit 1420.

Pulse wave signal normalization unit 140 normalizes a plurality of pulse wave signals obtained from the same subject extracted from the pulse wave extraction unit 130, to reduce a variation in wave pulse signals wherein the variation is shown per each number of the measurement of the same subject.

Pulse wave amplitude detection unit 150 divides pulse wave signal inputted to pulse wave extraction unit 130 or normalized pulse wave signal of the subject into a plurality of window sections and detects a minimum amplitude pulse (MIAP) and a maximum amplitude pulse (MXAP) respectively per each window section.

Personal pattern generating unit 1410 generates personal feature patterns using a minimum amplitude value in each window section detected by the pulse wave amplitude detection unit 150.

A storing unit 1420 stores personal feature pattern information generated by the personal pattern generating unit 1410 in a feature information section. In addition, as explained in detail above when a plurality of pulse wave signals are intended to be obtained from the same subject in order to normalize pulse wave signals, pulse wave signals extracted from the pulse wave extraction unit 130 are stored in a pulse wave signal storing section of the storing unit 1420.

Personal feature variation diagnostic unit 1430 diagnoses personal feature variation by using personal feature pattern stored in a feature information section of the storing unit 1420 and cuff pressure of the cuff 110 wound at the arm or wrist of the subject to compare measured feature patterns.

Display unit 180 can indicate personal feature information diagnosed in personal feature variation diagnostic unit 1430 such as high blood pressure data, kidney diseases and diabetes of the subject.

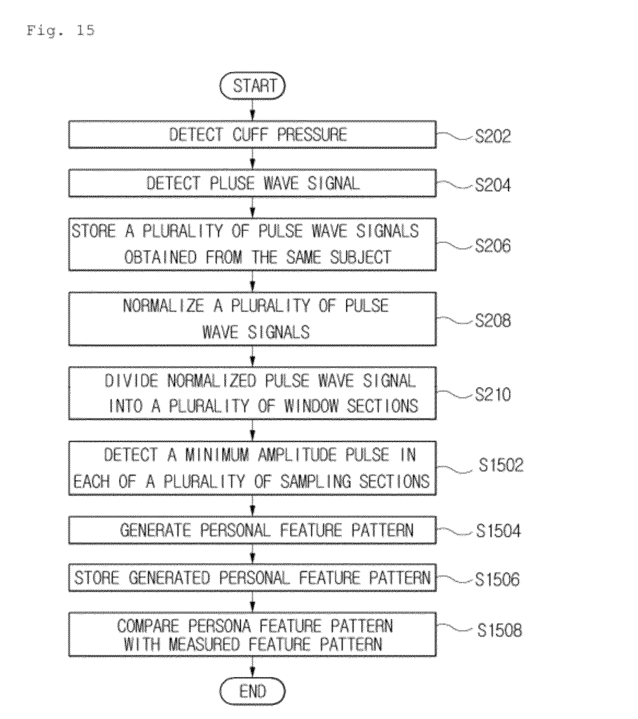

FIG. 15 is a flow chart which indicates personal feature diagnosing method based on an osillometric arterial pressure measurement shown in FIG. 14.

Cuff 110 is wound on arms or wrists and if a personal feature diagnostic unit 100 operates to measure a blood pressure a cuff pressure is detected via a pressure detection sensor unit 120 (S202).

Pulse wave signals of the artery in all sections are extracted at pulse wave extraction unit 130 from a cuff pressure detected from the pressure detection sensor unit 120 (S204).

Pulse wave signals extracted from pulse wave extraction unit 120 can be stored in a storing unit 1420 until a plurality of pulse wave signals are obtained from the same subject (S206).

If the measurement number for the same subject reaches N number, a pulse wave signal normalization unit 140 performs normalization in order to reduce a variation in pulse wave signals showing a variation per each measurement number of the same subject (S208).

Pulse wave amplitude detection unit 150 divides all sections of normalized pulse wave signal obtained from pulse wave signal normalization unit 140 into a plurality of window sections (S210).

In addition, pulse wave amplitude detection unit 150 detects minimum amplitude values each in a plurality of window sections (S1520).

Personal pattern generating unit 1420 generates personal feature patterns using a minimum amplitude value in window section detected by the pulse wave detection unit 150 (S1504). Preferably, personal pattern generating unit 1410 generates personal feature pattern by averaging out minimum amplitude values in each window section detected by pulse wave amplitude detection unit 150 per the corresponding window section for each subject.

Personal feature patterns generated in personal pattern generating unit 1410 are stored in the storing unit 1420 (S1506).

Furthermore, Personal feature variation diagnostic unit 1430 diagnoses personal feature variation by comparing personal feature pattern with feature pattern measured by using cuff pressure of cuff 110 wound on the arm of the subject (S1508).

Although the embodiments of this invention have been illustratively described herein, it will be apparent to a person skilled in the art that many changes or modifications may be made to the invention described above without departing from the spirit or scope of the appended claims. Therefore, it is intended that this invention not be limited to the particular embodiments disclosed as the best mode contemplated for carrying out the invention, but this invention includes all embodiments falling within the scope of the appended claims.

1. Personal feature diagnostic apparatus the basis of an oscillometric arterial pressure, comprising:
a pressure detection sensor unit for detecting a cuff pressure including an effect of pulse wave;
a pulse wave extraction unit for extracting pulse wave signals of the artery from the cuff pressure detected in the pressure detection sensor unit;
a pulse wave amplitude detection unit for dividing pulse wave signals extracted from the pulse wave extraction unit into a plurality of window sections and detecting a minimum amplitude pulse in each window section; and
a personal feature diagnostic unit for diagnosing personal feature by using the minimum amplitude pulse in each window section detected by the pulse wave amplitude detection unit.

2. The apparatus of claim 1, further comprising a pulse wave signal normalization unit for generating normalized pulse wave signals by using a plurality of pulse wave signals extracted from the pulse wave extraction unit obtained from the same subject to output the normalized pulse wave signals into the pulse wave amplitude detection unit wherein
the pulse wave amplitude detection unit divides the normalized pulse wave signals outputted from the pulse wave signal normalization unit into a plurality of window sections and detects a minimum amplitude pulse in each window section.

3. The apparatus of claim 2, wherein the personal feature diagnostic unit diagnoses personal features by averaging out a minimum amplitude pulse in each window section detected from the pulse wave amplitude detection unit per the corresponding window section per each subject.

4. The apparatus of claim 2, wherein the pulse wave signal normalization unit normalizes by using root value of sum of square of each of a plurality of pulse wave signals extracted from the pulse wave extracting unit.

5. The apparatus of claim 4, wherein the number of window at the time of dividing pulse wave signals extracted from the pulse wave extraction unit includes a minimum oscillometric waveform number and a pulse number of one turn in a maximum period.

6. The apparatus of claim 5, wherein the personal feature diagnostic unit diagnoses the subject as having a high blood pressure if the largest value of a minimum amplitude pulse or an average minimum amplitude pulse in each window section extracted by the pulse wave amplitude detection unit is within ⅓ of a measurement period.

7. The apparatus of claim 1, wherein the personal feature diagnostic unit diagnoses personal features using a minimum amplitude pulse and a maximum amplitude pulse in each window section detected by the pulse wave amplitude detection unit.

8. A personal feature diagnosing method on the basis of an oscillometric arterial pressure measurement, comprising steps of:
detecting a cuff pressure including an effect of pulse wave from a cuff wound on the arm or the wrist of the subject;
extracting pulse wave signals of the artery from the extracted cuff pressure;
dividing the extracted pulse wave signals into a plurality of window sections and detecting a minimum amplitude pulse in each window section; and diagnosing personal features using a minimum amplitude pulse in the each window section.

9. The method of claim 8 further comprising generating and extracting a normalized pulse wave signal using the plurality of pulse wave signals extracted from the same subject wherein the step of detecting a minimum amplitude pulse in the each window section comprises detecting a minimum amplitude pulse using the normalized pulse wave signal.

10. The method of claim 9, wherein the step of diagnosing the personal feature comprises diagnosing personal features by averaging out minimum amplitude pulses in each window section obtained per each subject per a corresponding window section.

11. The method of claim 8, wherein the step of diagnosing personal features comprises diagnosing personal features by using a minimum amplitude pulse and a maximum amplitude pulse in the each window section.

12. Personal feature diagnostic apparatus on the basis of an oscillometric arterial pressure, comprising:
a pressure detection sensor unit for detecting a cuff pressure including an effect of pulse wave;
a pulse wave extraction unit for extracting pulse wave signals of the artery from the cuff pressure detected in the pressure detection sensor unit;
a pulse wave amplitude detection unit for dividing pulse wave signals extracted from the pulse wave extraction unit into a plurality of window sections and detecting a minimum amplitude pulse in each window section;
a personal pattern generating unit for generating personal pattern by using the minimum amplitude pulse in each window section detected by the pulse wave amplitude detection unit;
a storing unit for storing personal feature pattern generated by the personal pattern generating unit; and
a personal feature variation diagnostic unit for diagnosing personal feature variation by comparing personal feature pattern stored in the storing unit with feature pattern measured using cuff pressure of cuff wound on the arm or wrist of the subject.

13. The apparatus of claim 12, further comprising a pulse wave signal normalization unit for generating a normalized pulse wave signal of the subject to output the normalized pulse wave signal to the pulse wave amplitude detection unit by using a plurality of pulse wave signals obtained from the same subject and extracted from the pulse wave extracting unit.

14. A personal feature diagnosing method on the basis of an oscillometric arterial pressure measurement, comprising steps of:
detecting a cuff pressure including an effect of pulse wave from a cuff wound on the arm or the wrist of the subject;
extracting pulse wave signals of the artery from the extracted cuff pressure;
dividing the extracted pulse wave signals into a plurality of window sections and detecting a minimum amplitude pulse in each window section;
generating personal feature pattern using a minimum amplitude pulse in the each window section;
storing the generated personal feature pattern; and
diagnosing personal feature variation by comparing the stored personal feature pattern with feature pattern measured using cuff pressure of cuff wound on the arm or wrist of the subject.
